(19) United States
(12) Patent Application Publication
(10) Pub. No.: US 2010/0057060 A1
(43) Pub. Date: Mar. 4, 2010

(54) In Situ UV/Riboflavin Ocular Treatment System

(75) Inventor: Satish V. Herekar, Palo Alto, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Seros Medical, LLC, Los Altos, CA (US)

(21) Appl. No.: 12/617,069

(22) Filed: Nov. 12, 2009

Related U.S. Application Data
(63) Continuation-in-part of application No. 12/273,444, filed on Nov. 18, 2008.
(60) Provisional application No. 61/118,897, filed on Dec. 1, 2008.
(60) Provisional application No. 61/012,333, filed on Dec. 7, 2007.

(51) Int. Cl. A61F 9/00

(52) U.S. Cl. 606/4

(57) ABSTRACT

A system for accurately delivering bilateral simultaneous equi-dosed time-fractionated pulsed UVA to irradiate a class of riboflavin/collagen mixture in the presence of oxygen for treatment of ocular tissue such as scleral and corneal tissue. The system employs ocular trial frames for mounting on the face that are fitted with 1) a nozzle for introducing Riboflavin in solution to collagen on the surface of the ocular tissue, 2) a port for introducing oxygen-rich gas to the ocular tissue, and 3) a pair of optical collimator inserts mounted in the lens holders, wherein the collimator inserts have a mask in the optical path at an aperture on focal point to control the pattern of UVA radiation at the ocular target, the collimator inserts further having optical input ports coupled to a controlled source of UVA radiation that is operative in accordance with the related inventive method.

### CROSS-REFERENCES TO RELATED APPLICATIONS

The present application claims benefit under 35 USC 119(e) of U.S. Provisional Application Ser. No. 61/118,897 filed Dec. 1, 2008.

The present application is also a continuation-in-part of co-pending U.S. application Ser. No. 12/273,444, filed on Nov. 18, 2008, entitled “Method For Equi-Dosed Time Fractionated Pulsed UVA Irradiation Of Collagen/Riboflavin Mixtures For Ocular Structural Augmentation” (published Patent Publication US-2009-0149923-A1 of Jun. 11, 2009), the content of which is incorporated herein by reference, The foregoing application in turn claims benefit under 35 USC 119(e) of U.S. Provisional Application Ser. No. 61/012,333 filed Dec. 7, 2007.

### STATEMENT AS TO RIGHTS TO INVENTIONS MADE UNDER FEDERALLY SPONSORED RESEARCH OR DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

The present invention relates generally to an instrument for use in treating ocular tissue. More particularly, the invention relates to a treatment delivery apparatus for augmenting corneal, scleral and retinal ocular tissue and for treating, such as by repairing and reshaping, ocular tissues and eventually for refractive surgery.

Corneal and other ocular structural weakness, such as scleral structural weaknesses, can have several origins, including genetic, iatrogenic, accidents and shortcoming of desired surgical correction. Furthermore, ulcerations, melts and the like may require localized repair. Refractive corrections may comprise corneal reshaping surgery or addition of prosthetics (inlays/onlays/cavity augmentations) or some combination thereof. Localized repair is currently performed by lamellar surgery, which requires precise in situ “fitting” of biocompatible host and donor tissues and maintenance of smooth interfaces and biocompatibility thereafter, all of which are not insignificant issues. Complications from laser-based surface shaving surgery are well-known. Suturing has its own set of difficulties and shortcomings, as does tissue gluing.

In the above-referenced co-pending non-provisional patent application, a method is taught for effectively treating ocular tissue using collagen exposed to riboflavin, also known as vitamin B2, in the presence of ultraviolet light to produce cross-linking, which is useful as a cell scaffold for rebuilding cartilaginous defects. The new technique involves irradiating collagen in situ with target ocular tissue by time-fractionated pulsed UVA irradiation in the presence of riboflavin. However, problems exist with known delivery systems due to the lack of control over the positioning of the eye with respect to the delivery system.

In work by the present inventor (not as prior art) identified in Provisional Patent Application No. 60/869,048 filed Dec. 7, 2006, and now found in Non-Provisional Patent Application Ser. No. 11/952,801 filed Dec. 7, 2007, as well as in the aforementioned Non-Provisional Patent Application Ser. No. 12/273,444, filed Nov. 18, 2008, and its priority Provisional Patent Application No. 61/012,333 filed Dec. 7, 2007, various methods and materials for in situ corneal structural augmentation were disclosed involving irradiation of collagen/riboflavin mixtures. The present invention is useful in such treatment.

### SUMMARY OF THE INVENTION

According to the invention, a system is for provided for accurately delivering bilateral simultaneous equi-dosed time-fractionated pulsed UVA to irradiate a class of riboflavin/collagen mixture in the presence of copious oxygen to cause rapid cross-linking resulting in gelation of the riboflavin/collagen mixture in situ and to effect adhesion to underlying structure, specifically ocular tissue such as scleral and corneal tissue. The system according to an embodiment of the invention comprises ocular trial frames for mounting on the face that are fitted with 1) a nozzle for introducing Riboflavin in solution to collagen on the surface of the ocular tissue, 2) a port for introducing oxygen-rich gas to the ocular tissue, and 3) a pair of optical collimator inserts mounted in the lens holders, wherein the collimator inserts have a mask in the optical path at an aperture on focal point to control the pattern of UVA radiation at the ocular target, the collimator inserts further having optical input ports coupled to a controlled source of UVA radiation that is operative in accordance with the related inventive method. The device promotes bilateral simultaneous treatment of specifically targeted collagen enhanced ocular tissue with UVA radiation in the presence of Riboflavin and oxygen. An intended application is structural augmentation of ocular tissue, as may be used for better stabilizing progressive corneal diseases, such as keratoconus (KCN), ectasia, ulcers/melts and the like.

Accurate patterned pulsed UVA radiation, delivers significantly stronger (at depth) and safer collagen cross-linking in a shorter time.

The disclosed system, which comprising a sterilizable ocular trial frame with add-on optics and fluid or drug delivery modals, provides for ocular exposure technique for corneo/scleral/retinal delivery by conventional cross-linking (XL), cross-linking with augmentation (XLA), rapid cross-linking or high-intensity cross-linking (RXL), pulsed cross-linking or high frequency UVA cross-link (PXL), and fractionated cross-linking or UVA exposure pauses (FXL) for treatments of, among typical conditions, keratoconus, ectasia, post-op stabilization, progressive myopia, augmentation, ulcers, PMD, melts, bullous keratopathy (BK) and anti-bacterial infections.

The invention will be better understood by reference to the following drawing and related description.

### BRIEF DESCRIPTION OF THE DRAWINGS

FIG. 1 is a perspective diagram of a headpiece for delivering corneal augmentation according methods related to the invention.

FIG. 2 is a cross-sectional view showing elements of the invention.

FIGS. 3A and 3B are representative masks according to the invention.

### DETAILED DESCRIPTION OF THE INVENTION

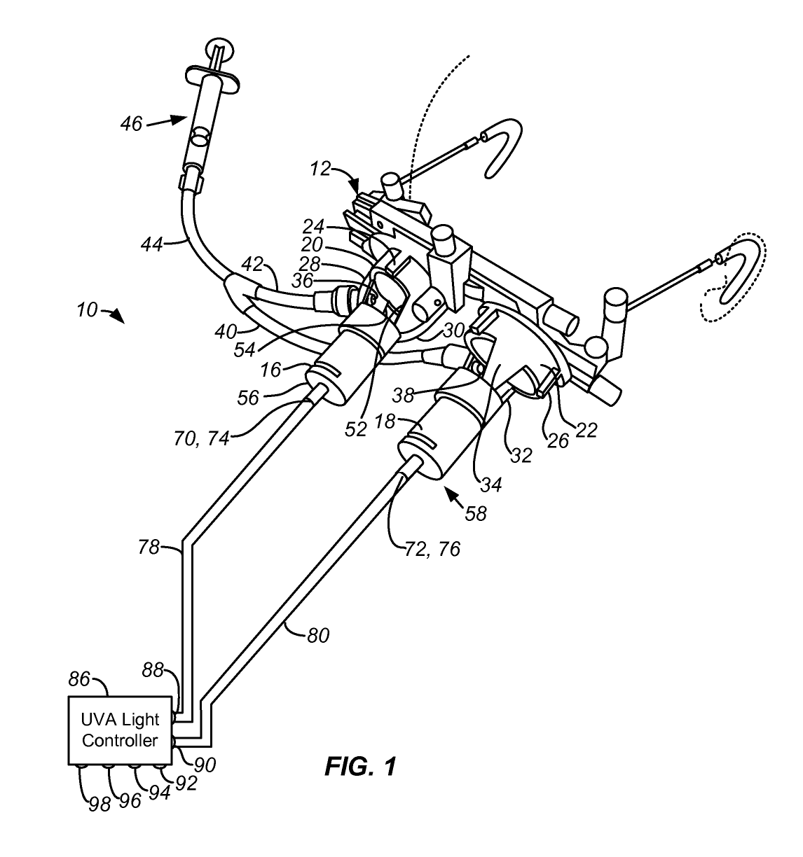

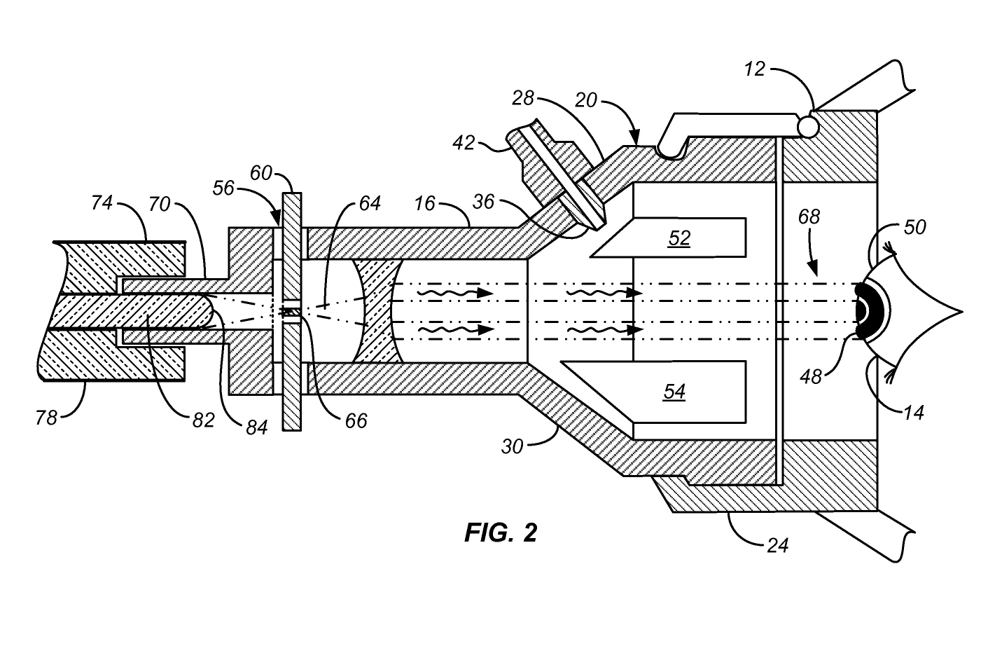

According to a specific embodiment of the invention, and referring to FIGS. 1 and 2, a system 10 for ocular treatment including ocular trial frames 12 for mounting on the face and disposed to fit over the eyes 14. The optical trial frames are fitted with a pair of optical collimators 16, 18 having an end mounts 20, 22 that interlock with the lens holders 24, 26 of the trial frames 12. Arms 28, 30, 32, 34 connect the tubes of the collimators to the end mounts 20, 22. The arms 28, 30, 32, 34 define a semi-enclosed region for treatment. The arms are sufficiently broad to be fitted with one or more nozzles 36, 38 for introducing Riboflavin in solution via tubing 40, 42, 44 to an injector 46 to collagen 48 applied to the surface of the ocular tissue 40. The nozzles 36, 38 may be fitted to employ standard luer locks. The same structure can be used to deliver topical anesthetics, photosensitizers, crosslinkers, catalysts and other biomaterials or medications as needed. Between the arms are openings or ports for introducing oxygen-rich gas to the ocular tissue. The oxygen source may be ambient air; thus the ports are large, or there may be a separate oxygen source coupled via tubing to nozzles similar to those of the nozzles 36, 38 to supply oxygen gas, heated, cooled, humidified or dehumidified gas or air, to the ocular tissue. Moreover the gas ports and the fluid ports may be structured to be interchangeable.

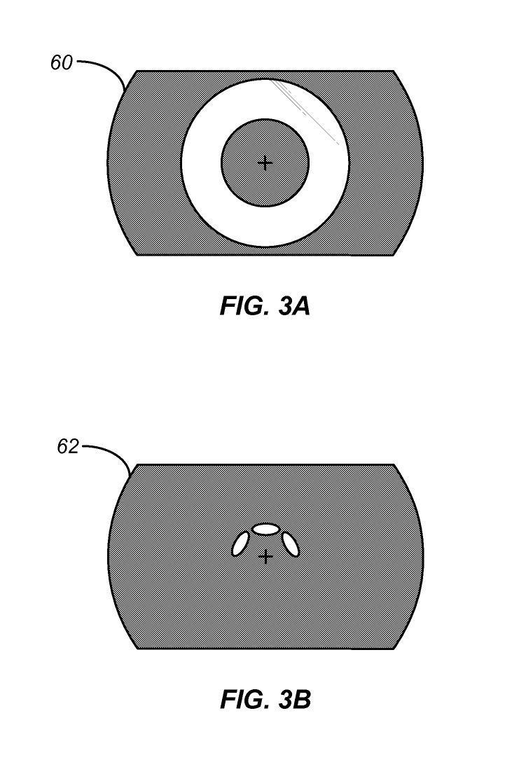

Each of the optical collimator inserts 16, 18 has a mask holder 56, 58 in which various masks 60, 62 (FIGS. 2 and 3A and 3B) may be mounted in the optical path 64 at an aperture 66 on focal point to control the pattern of UVA radiation at the ocular target. The mask 60 is for blocking out radiation directed at the ocular lens region and therefore has a pass through that is annular in shape. The mask 62 is for passing several columns of radiation that are spatially separated. The collimator inserts 16, 18 further have optical input ports 70, 72 which engage the couplings 74, 76 on the ends of fiber optic cables 78, 80 in which an optical fiber 82 is embedded.

Referring to FIG. 2, the end of the optical fiber 82 is fitted with a focusing lens end 84 to focus UVA radiation at the focal point at the aperture 66. The aperture 66 produces an image in the target region 68 of the eye, the mask having a pattern matching that of the masks 60, 62 but impinging on the collagen material 48 that has been placed on the ocular tissue. Because of imaging of the fiber tip on the eye, the spatial pattern of UV on the corneal and/or scleral tissue can be controlled quite accurately.

The input ends of each optical fiber cable 78, 80 are coupled to a controller 86, which comprises a source of UVA radiation, such as a UVA laser, that is operative in accordance with the related inventive method. UVA output ports 88, 90 receive the optical fibers 78, 80. Because fiber coupling is used, the uniformity of the beam can be more closely controlled. The controller 86 is provided with adjustment elements for treatment duration 92, duty cycle of the UVA 94 to produce time fractionated output, radiation intensity 96 and pulse duration 96. Pulse duration and intensity may be preset based on any calibration considerations.

The collagen/riboflavin mixture that is produced by the placement of the collagen 48 and the injection of fluid in drops or mist through the nozzles 36, 38 is irradiated with UVA radiation in a specific timing pattern as specified by the controller 86 and spatial pattern as dictated by the selected mask 60 in the mask holder 56. A specific pattern of pulses at a fractionated duty cycle in the presence of oxygen targeting each eye simultaneously generates reactive oxygen species and to cause desired forms of gelation, that is, gelation with robustness in terms of stability, longevity, rigidity, optical clarity, low shrinkage and high adhesion to a substrate of ocular tissue. Decreasing the time of UVA exposure or minimizing the UVA intensity in a therapy according to the invention tends to minimize undesired cellular changes during augmentation or generation of in situ collagen gels.

Modulating exposure affects the nature of the gels formed. Using equi-dosed conditions as a modus, UVA is applied in time-fractionated pulses to collagen/riboflavin mixtures in the form of amorphous gels. Collagen/riboflavin mixtures that were prepared as previously described in Provisional Patent Application 60/869,048 filed Dec. 7, 2006, and its corresponding Non-Provisional Patent Application entitled “Method And Material For In Situ Corneal Structural Augmentation” in the name of the present inventor (U.S. patent application Ser. No. 11/952,801) using a 6% bovine collagen solution at a pH of 5.5 and 6.5 containing riboflavin-based cross-linker in a ratio of 5:100, although a wide range of concentration mixtures is contemplated and have a significant effect on rapidity of gelation.

According to an embodiment of the present invention, the collagen/riboflavin mixture is rapidly gelated to an intended robustness by exposing the mixture in the presence of oxygen to a fractionated dosage of pulsed UVA directed bilaterally through the collimators 16, 18, thereby generating reactive oxygen species of singlet oxygen that has beneficial outcomes. The UVA is at an instantaneous fluence (intensity per sq. cm.) of between 1 mW/cm² and 30 mW/cm² and preferably at a nominal optimal value of 15 mW/cm² during the ON portion of the duty cycle, which may vary from 1% to 100% for experimental purposes but less than 100% for actual operation and preferably for a period of a few seconds on at a nominal optimal duty cycle of 20% or 1:5 with OFF time of approximately 30 seconds over a period of 6 minutes. However, a duty cycle of between 2:1 (50% on) and 3:1 (67% on) with off time of about 30 seconds over a period of 12 minutes has been employed effectively in experiment.

In use, the therapist user or surgeon has visual access during treatment or surgery, and the device has both unilateral and bilateral simultaneous capability with homogenous, top hat and alignment-tolerant beam delivery with better patient comfort/interface. This results in improved treatment accuracy and provides for patient customizable parameters, such as projected pattern selection by means of various masks. The masks shown in FIGS. 3A and 3B are merely suggestive. Other suitable patterns, besides annular and spot, are bowtie and a variety of spot sizes (up to about 12 mm and shapes to match the area of intended exposure. Other controllable parameters are pupillary distance, vertex control and the like (as provided by state-of-the-art ocular trial frames), wavelength selection (blue/UVA etc) and importantly, programmable irradiance, exposure duration, selection of continuous or pulsed timed exposures, which can be administered singly or simultaneously for each eye.

The system may be provided with a manual feedback mechanism, such as a visual display that is connected to a controller and sensors to monitor optical input, medication delivery and the like. It may likewise be equipped with safety systems to disable the system and warn the user about undesired or unsafe conditions.

Although the system has been shown with dual optical sources and a single fluid delivery system, it is not a departure from the invention to provide a single optical source and a dual fluid delivery system.

The invention has been explained with respect to specific embodiments and examples. Other embodiments will be evident to those of skill in the art. It is therefore not intended that the invention be limited, except in accordance with the appended claims.

What is claimed is:

1. A system for rapid bilateral simultaneous treatment of ocular tissue in connection with a source of ultraviolet radiation comprising:
a pair of ocular trial frames having lens mounts;
first and second collimators mounted in first and second holders, each disposed in front of each eye position to the frames for mounting to the head and aligning with both eyes;
first and second nozzle mounted to respective first and second holders for introducing riboflavin in solution into contact with collagen on ocular tissue of the eyes to yield a collagen/cross-linker mixture
first and second openings in respective first and second holders for admitting oxygen into contact with the collagen/cross-linker mixture in the eyes; and
first and second optical input port for the first and second collimators for receiving the ultraviolet radiation.

2. A system according to claim 1 further including:
first and second mask holders for first and second masks disposed in the optical paths of the respective first and second collimators.

3. A system according to claim 1 further including:
first and second fiber optic conduits coupled to the first and second input ports;
an ultraviolet radiation source for supplying ultraviolet radiation to the first and second fiber optic conduits; and
a controller coupled to the ultraviolet source for controlling simultaneous bilateral irradiance in equi-dosed time fractionated pulses at a selected fractionated duty cycle over a selected exposure period sufficient to yield gelation with desired physical characteristics.

4. A system according to claim 3 further including:
first and second mask holders for first and second masks disposed in the optical paths of the respective first and second collimators.

5. A method for rapid bilateral simultaneous treatment of ocular tissue comprising effecting gelation of a collagen/riboflavin mixture having a controlled mixture ratio and concentration in situ on ocular tissue for enhancement of said ocular tissue, the method comprising:
applying collagen to ocular tissue in situ;
introducing riboflavin in solution into contact with the collagen to yield a collagen/cross-linker mixture;
irradiating each eye, though a pair of collimators mounted on frames mounted to the head and aligned with the eyes, the collagen/cross-linker mixture in the presence of oxygen with ultraviolet energy in equi-dosed time-fractionated pulses over an exposure period sufficient to promote the generation of reactive oxygen species and to yield gelation with desired physical robustness and adhesion to said ocular tissue.

6. The method according to claim 5 wherein the ultraviolet energy is UVA radiation.

7. The method according to claim 6 wherein the irradiation is in a preselected spatial pattern to impinge only on restricted regions of the ocular tissue.

8. The method according to claim 7 wherein the spatial pattern is defined by a mask in the path of the UVA radiation.

9. The method according to claim 8 wherein the spatial pattern defined by a mask is annular having a central blockage.

10. The method according to claim 8 wherein the spatial pattern defined by a mask is of plural selected spots corresponding to locations of the collagen cross-linker mixture.

11. The method according to claim 7 wherein the instantaneous fluence of said UVA is between 1 mW/cm² and 30 mW/cm².

12. The method according to claim 11 wherein treatment fractionation has an on/off duty cycle of between 1:100 and 100:1.

13. The method according to claim 11 wherein said UVA instantaneous fluence is less than 15 mW/cm² at 355 nm frequency and the pulses have pulse rate at a frequency
of greater than 100 kHz with greater than a 1:3 duty cycle for a treatment fractionation duty cycle of greater than 1:5 for a treatment duration of less than 6 minutes.

14. The method according to claim 13 wherein the irradiance exposure off time is less than 30 seconds.

15. The method according to claim 13 wherein treatment fractionation has an on/off duty cycle of between 2:1 and 3:1.

16. The method according to claim 15 wherein the irradiance exposure off time is less than 30 seconds.

17. The method according to claim 7 wherein treatment fractionation has an on/off duty cycle of approximately 1:5.
